(19) United States
(12) Patent Application Publication
(10) Pub. No.: US 2022/0072333 A1
(43) Pub. Date: Mar. 10, 2022

(54) MYOPIA-PREVENTING ILLUMINATION APPARATUS FOR ILLUMINATING EYEBALLS AND SURROUNDING TISSUES VIA LIGHT THAT PENETRATING PERIORBITAL SKIN, SUBCUTANEOUS TISSUE,THEN PASS THROUGH CORNEA, IRIS, UVEA, SCLERA AND CHOROID

(71) Applicants: CHWEN-YIH LIN, New Taipei City (TW); YU-HAO LIN, New Taipei City (TW)

(72) Inventors: CHWEN-YIH LIN, New Taipei City (TW); YU-HAO LIN, New Taipei City (TW)

(21) Appl. No.: 17/014,201

(22) Filed: Sep. 8, 2020

(51) Int. Cl. A61N 5/06

(52) U.S. Cl. CPC A61N 5/0622; A61N 2005/0626; A61N 2005/0648

(57) ABSTRACT

The present invention provides a myopia-preventing illumination apparatus for illuminating eyeballs and surrounding tissues via light that penetrating periorbital skin, subcutaneous tissue, then pass through cornea, iris, uvea, sclera and choroid, comprising a light source and a controller electrically connects to the light source, wherein the light source is projects to and illuminates on skin surrounding eyes, penetrates and enters subcutaneous tissue, iris, ciliary body and lens, as well as penetrates peripheral tissues of eyeballs, sclera, uvea, choroid and retinal pigment epithelium, and indirectly enters into vitreous and retina, so as to prevent myopia from further worsening by inducing microscopic biochemical reactions.

### BACKGROUND OF THE INVENTION

### a) Field of the Invention

The present invention relates to a myopia-preventing illumination apparatus for illuminating eyeballs and surrounding tissues via light that penetrating periorbital skin, subcutaneous tissue, then pass through cornea, iris, uvea, sclera and choroid, specifically a device and method of transcutaneous, transuveus, transsclera, transchoroid, illumination of eyes and periorbital region.

### b) Description of the Prior Art

Myopia is a worldwide problem, especially in Asia. Myopia tends to develop in childhood and progress in severity until adulthood. Myopia associates with many disadvantages such as inconvenient in work, cost for glasses or contact lenses as well as increasing eye diseases relate to myopia. Hence, the prevention of progression of myopia is a great health issue.

### PRIOR ARTS

One invention CN101858573A is a high illuminance classroom lamp, refer to apply high illuminance classroom lamp to help the students in preventing myopia. Another invention EP3141282A1 is a device for treating, preventing, or reducing myopia, or the risk thereof, use a display in front of student to apply various lights (illuminance of 0.6 to 1000 lux) to stimulate eyes. Another invention US20170072218A is a Method and apparatus for reducing or preventing myopia, apply a display to stimulate the temporal region of the eye to prevent myopia. Another invention EP2155041A1 is a determination of optical adjustments for retarding myopia progression, provides an anti-myopia lens to retard myopia progression. Another invention U.S. Pat. No. 9,709,826B2 is an ocular lens, apply ocular lens with filtered bands of wavelength to prevent myopia progression. Another invention WO2015152818A1 is a device to prevent a condition or disease associated with lack of outdoor time, apply a wearable device to monitor the outdoor time of children in order to prevent myopia progression. Another invention CN107707763A is a myopia prevention and control wearable device and myopia prevention and control system and method, apply a wearable device to monitor the reading illuminance and reading distance in order to prevent the progression of myopia. Another invention CN106289395A is a student myopia prevention and control wearable device, apply a wearable device with ambient light sensor and temperature sensor and laser distance sensor to monitor the student's reading pattern in order to prevent the myopia progression.

The effectiveness of the prior arts are not confirmed, yet the myopia rate all over the world is still high, hence, there is high demand for a effective myopia preventing method or device.

There are studies show that the time of daily outdoor activity is a factor for preventing the myopia progression, hence arose inventions focus on monitor of daily outdoor time and advice the student to adjust their activity for preventing myopia, other inventions focus on light stimulation either with display or with classroom light to stimulate eyes but due to the physiology of eyes, the pupil will constrict in response of high illumination, the stimulate effect is doubtful, besides, high direct illumination will induce macula disease.

Most classroom illumination standard is about 300-500 lux, while the intensity of outdoor illumination is easily arrive 50000 lux, so the intensity of outdoor illumination is higher than that of indoor, but an eye can't stand for long time of illumination more than 10000 lux directly into retina, the retina will be injured. So the pupils and eyelids are acting as a protection device to reduce the illumination directly introduce into retina. The light directly introducing into retina is greatly reduced in outdoor activity due to the active regulation of pupil and eyelids. But outdoor activity is effective in myopia prevention which is indicated in many studies, so the light pass through eyelids must play a great role.

The outdoor activity is different in that high illumination light applied directly on the skin in peripheral of eyes which transmit into periorbital tissue and penetrate into the eyeball indirectly, so the retina is not injured by the high illumination, while the transcutaneous light stimulate periorbital skin, subcutaneous tissue, cornea, iris, ciliary body, uveu, lens, sclera, periorbital tissue, choroid, retina pigment epithelium, retina and vitreous indirectly, which induce a subtle biochemical reaction that preventing the progression of myopia.

### SUMMARY OF THE INVENTION

The primary object of the present invention is to construct a device which mimic the outdoor illumination that illuminate directly into the periorbital skin, then penetrating into the subcutaneous tissue, and pass through cornea and the iris, ciliary body, lens, transmitting into periorbital tissue, pass through sclera, uvea, choroid, retina pigment epithelium and indirectly into retina and vitreous to induce the subtle biochemical reaction which preventing the progression of myopia.

### BRIEF DESCRIPTION OF THE DRAWINGS

FIG. 1 is the system block diagram of the present invention.

FIG. 2 is the schematic diagram of a usage state of the present invention.

FIG. 3 is the schematic diagram of the 1st structural assembly of the present invention.

FIG. 4 is the schematic diagram of the 2nd structural assembly of the present invention.

FIG. 5 is the schematic diagram of the 3rd structural assembly of the present invention.

FIG. 6 is the schematic diagram of the 4th structural assembly of the present invention.

FIG. 7 is the schematic diagram of the 5th structural assembly of the present invention.

FIG. 8 is the schematic diagram of the 6th structural assembly of the present invention.

FIG. 9 is the schematic diagram of the 7th structural assembly of the present invention.

FIG. 10 is the schematic diagram of the 8th structural assembly of the present invention.

### DETAILED DESCRIPTION OF THE PREFERRED EMBODIMENTS

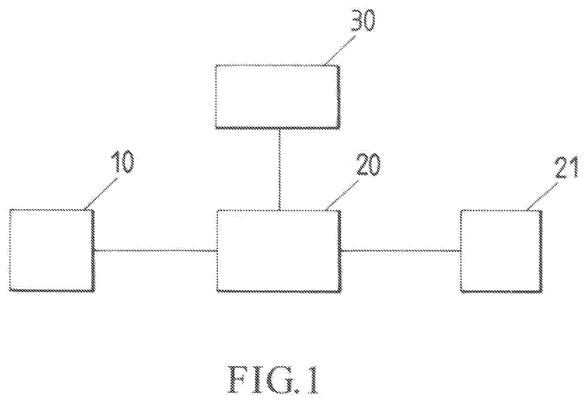

Referring to the FIG. 1, the present invention comprising a light source 10 and a controller 2, wherein the light source 10 comprises various spectra, and the spectra and compositions thereof can be adjusted according to the requirement; the light source 10 can be one type of light source or a composition made up of various light sources, and the illumination level provided by the light source 10 is adjustable and can be adjusted based on different time periods, and the illumination level is greater than 0.5 LUX or programmably adjusted; the illumination timing of the light source 10 can be continuous, intermittent, a mixture thereof or programmably adjusted, the illumination period and brightness level can be modified and adjusted on the basis of a clock 30, and the illumination time each day can be adjusted according to a requirement with respect to different myopic persons.

The controller 20 is a CPU and is electrically connected to the light source 10 and the clock 30, wherein the controller 20 is electrically connected to a battery 21, and the battery 21 supplies power required for the light source 10, the controller 20 and the clock 30.

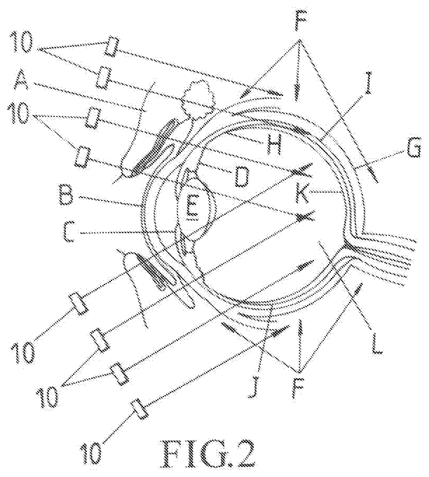

Referring to FIG. 2, at least one light source 10 of the present invention is projected to and illuminated on skin surrounding eyes, penetrates subcutaneous tissue A, then pass through cornea B, iris C, ciliary body D and lens E, or penetrates a peripheral tissue F of eyeball, sclera G, uvea H, choroid I, retinal pigment epithelium J, and indirectly enters into vitreous L and retina K, so as to prevent myopia from further worsening by inducing microscopic biochemical reactions.

The structure of the present invention has an unlimited appearance design, and can have various types of architectures:

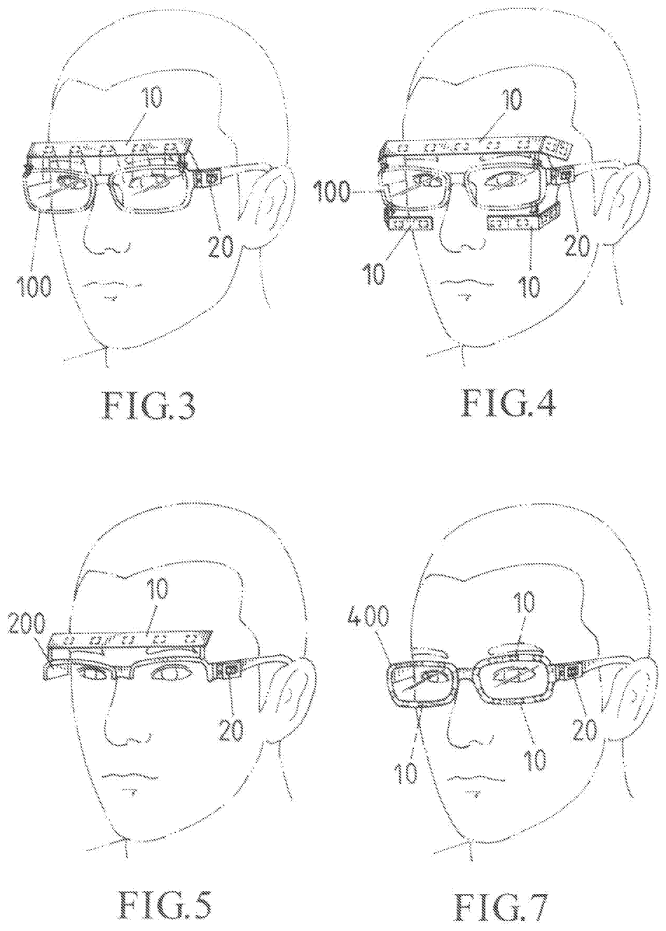

FIG. 3 is the 1st embodiment of the present invention, A light source 10 is provided above an existing pair of glasses 100, and the light source 10 can be clamped and fixed on the pair of glasses 100 by using a clamp, while a controller 20 is clamped and fixed next to the light source 10 and electrically connected to the light source 10.

FIG. 4 is the 2nd embodiment of the present invention, At least one light source 10 is clamped and fixed above, below or laterally to an existing pair of glasses 100, while at least one controller 20 is clamped and fixed next to the light sources 10 and electrically connected to the light sources 10.

FIG. 5 is the 3rd embodiment of the present invention, A vision corrected person wears an illumination device frame 200, wherein the illumination device frame 200 is provided thereon with at least one light source 10, while a controller 20 is provided next to the light source 10 and electrically connected to the light source 10; illumination provided by the light source 10 can be respectively and individually controlled, or collectively controlled by a unit related to a main program of the illumination device frame 200.

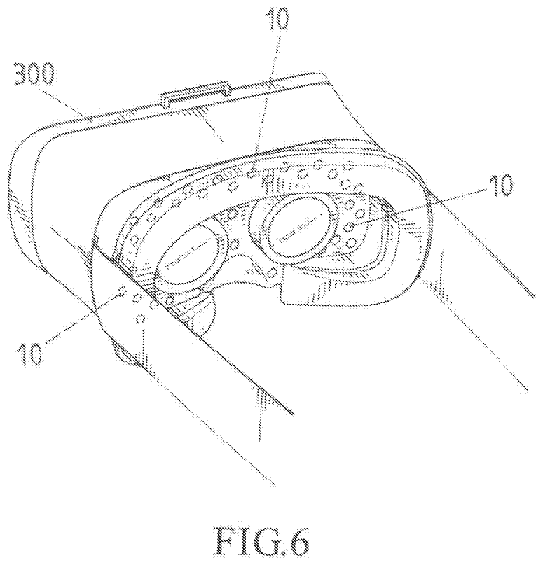

FIG. 6 is the 4th embodiment of the present invention, A vision corrected person wears a pair of virtual reality (VR) or augmented reality (AR) glasses 300, wherein the pair of VR or AR glasses 300 or a frame similar thereto is provided thereon with at a light source 10, while a controller 20 is provided next to the light source 10 and electrically connected to the light source 10; illumination provided by the light source 10 can be respectively and individually controlled, or collectively controlled by a unit related to a main program of the pair of VR or AR glasses 300 or the frame similar thereto.

FIG. 7 is the 5th embodiment of the present invention, It comprising a customized glasses frame 400, at least one light source 10 is directly provided on the glasses frame 400 so as to provide illumination, while a controller 20 is provided next to the light source 10 and electrically connected to the light source 10.

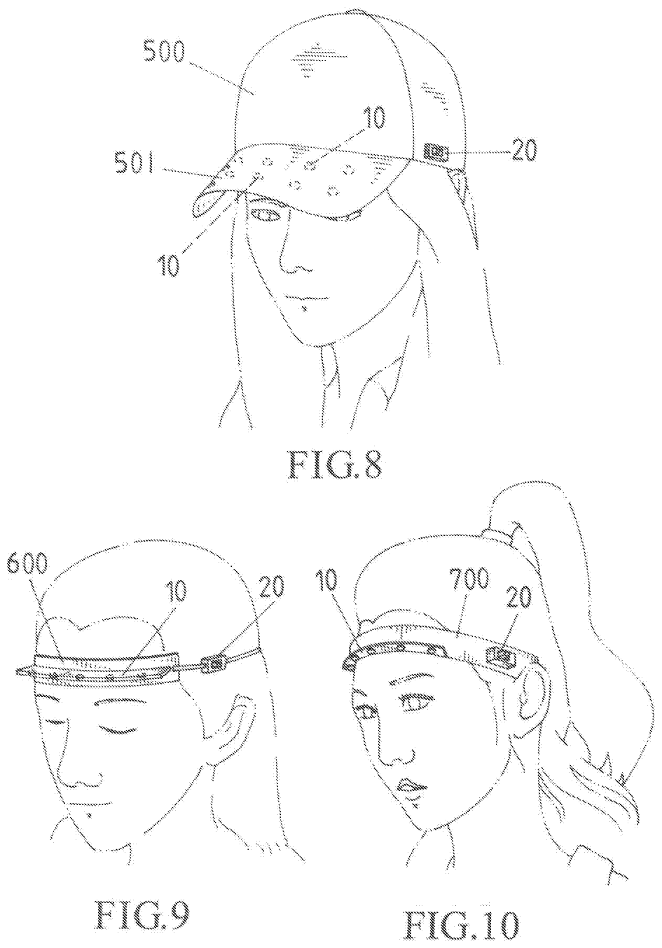

FIG. 8 is the 6th embodiment of the present invention, It comprising a customized headgear 500, and at least one light source 10 is provided on an edge 501 of the headgear 500, while a controller 20 is provided in an adequate position on the headgear 500, and the controller 20 is electrically connected to the light source 10.

FIG. 9 is the 7th embodiment of the present invention, It comprising a customized hair band or head band 600, and the hair band or head band 600 has a frontal edge thereof provided with a light source 10, while at least one controller 20 is provided next to the light sources 10 and electrically connected to the light sources 10.

FIG. 10 is the 8th embodiment of the present invention, It comprising a customized pad 700, and a light source 10 can be provided on the pad 700 on the head of a vision corrected person, while a controller 20 is provided in an adequate position on the pad 700, and the controller 20 is electrically connected to the light source 10.

In summary, the present invention is to construct a device which mimic the outdoor illumination that illuminate directly into the periorbital skin and penetrating into the subcutaneous tissue, and pass through cornea and the iris, ciliary body, lens, or transmitting into periorbital tissue, pass through sclera, uvea, choroid, retina pigment epithelium and indirectly into vitreous and retina to induce the subtle biochemical reaction which preventing the progression of myopia.

It is of course to be understood that the embodiments described herein are merely illustrative of the principles of the invention and that a wide variety of modifications thereto may be effected by persons skilled in the art without departing from the spirit and scope of the invention as set forth in the following claims.

1. A myopia-preventing illumination apparatus for illuminating eyeballs and surrounding tissues via light that penetrating periorbital skin, subcutaneous tissue, then pass through cornea, iris, uvea, sclera and choroid, comprising a light source and a controller electrically connects to the light source, wherein the light source is projects to and illuminates on skin surrounding eyes, penetrates and enters subcutaneous tissue, iris, ciliary body and lens, as well as penetrates peripheral tissues of eyeballs, sclera, uvea, choroid and retinal pigment epithelium, and indirectly enters into vitreous and retina, so as to prevent myopia from further worsening by inducing microscopic biochemical reactions.

2. The myopia-preventing illumination apparatus for illuminating eyeballs and surrounding tissues via light that penetrating periorbital skin, subcutaneous tissue, then pass through cornea, iris, uvea, sclera and choroid according to claim 1, wherein the light source comprises various spectra, wherein the spectra and compositions thereof can be adjusted according to a requirement, the light source can be one type of light source or a composition of various light sources, the illumination level provided by the light source is adjustable, and being adjusted to greater than 0.5 LUX or programmably adjusted based on different time periods; the illumination timing of the illumination provided by the light source being continuous, intermittent, a mixture thereof or programmably adjusted, and the illumination period and the brightness level of the illumination provided by the light source can be modified and adjusted on the basis of a clock.

3. The myopia-preventing illumination apparatus for illuminating eyeballs and surrounding tissues via light that penetrating periorbital skin, subcutaneous tissue, then pass through cornea, iris, uvea, sclera and choroid according to claim 1, wherein the controller is a CPU and is electrically connects to the light source, the clock and a battery, wherein the battery supplies power required for the light source, the controller and the clock.

4. The myopia-preventing illumination apparatus for illuminating eyeballs and surrounding tissues via light that penetrating periorbital skin, subcutaneous tissue, then pass through cornea, iris, uvea, sclera and choroid according to claim 1, wherein the light source is provided above, below or laterally to a pair of glasses.

5. The myopia-preventing illumination apparatus for illuminating eyeballs and surrounding tissues via light that penetrating periorbital skin, subcutaneous tissue, then pass through cornea, iris, uvea, sclera and choroid according to claim 1, wherein the light source is provided on an illumination device frame, wherein illumination provided by the light source being respectively and individually controlled, or collectively controlled by a main program of the illumination device frame.

6. The myopia-preventing illumination apparatus for illuminating eyeballs and surrounding tissues via light that penetrating periorbital skin, subcutaneous tissue, then pass through cornea, iris, uvea, sclera and choroid according to claim 1, wherein the light source is provided on a pair of VR or AR glasses or a frame similar thereto, wherein illumination provided by the light source being respectively and individually controlled, or collectively controlled by a main program of the pair of VR or AR glasses or the frame similar thereto.

7. The myopia-preventing illumination apparatus for illuminating eyeballs and surrounding tissues via light that penetrating periorbital skin, subcutaneous tissue, then pass through cornea, iris, uvea, sclera and choroid according to claim 1, wherein the light source is directly provided on a glasses frame.

8. The myopia-preventing illumination apparatus for illuminating eyeballs and surrounding tissues via light that penetrating periorbital skin, subcutaneous tissue, then pass through cornea, iris, uvea, sclera and choroid according to claim 1, wherein the light source is provided on an edge of a headgear.

9. The myopia-preventing illumination apparatus for illuminating eyeballs and surrounding tissues via light that penetrating periorbital skin, subcutaneous tissue, then pass through cornea, iris, uvea, sclera and choroid according to claim 1, wherein the light source is provided on a hair band or head band.

10. The myopia-preventing illumination apparatus for illuminating eyeballs and surrounding tissues via light that penetrating periorbital skin, subcutaneous tissue, then pass through cornea, iris, uvea, sclera and choroid according to claim 1, wherein the light source is provided on a pad, wherein the pad being adhered to the head of a vision corrected person.
